(19) United States
(12) Patent Application Publication
(10) Pub. No.: US 2024/0350008 A1
(43) Pub. Date: Oct. 24, 2024

(54) INTRAOCULAR PRESSURE SENSOR DEVICE AND METHOD

(71) Applicants: NATIONAL UNIVERSITY OF SINGAPORE, Singapore (SG); NATIONAL UNIVERSITY HOSPITAL (SINGAPORE) PTE LTD, Singapore (SG)

(72) Inventors: Chee Keong TEE, Singapore (SG); Kelu YU, Singapore (SG); Si LI, Singapore (SG); Victor KOH, Singapore (SG); Kwok Hung LEE, Singapore (SG)

(73) Assignees: NATIONAL UNIVERSITY OF SINGAPORE, Singapore (SG); NATIONAL UNIVERSITY HOSPITAL (SINGAPORE) PTE LTD, Singapore (SG)

(21) Appl. No.: 18/685,009

(22) PCT Filed: Aug. 22, 2022
(86) PCT No.: PCT/SG2022/050598

§ 371 Date: Feb. 20, 2024

(30) Foreign Application Priority Data
Aug. 20, 2021 (SG) 10202109128P

(51) Int. Cl. A61B 3/16

(52) U.S. Cl. CPC A61B 3/16

(57) ABSTRACT

A system and method for measuring intraocular pressure (IOP) of the eye. The method 5 comprises the steps of touching the eyelid with a pressure sensor array; obtaining a spatiotemporal representation of pressure sensor stimulation of the pressure sensor array while touching the eyelid with the pressure sensor array; and applying a machine learning model to classify the spatiotemporal representation into an IOP value.

### FIELD OF INVENTION

The present invention relates broadly to an intraocular pressure sensor device and method.

### BACKGROUND

Any mention and/or discussion of prior art throughout the specification should not be considered, in any way, as an admission that this prior art is well known or forms part of common general knowledge in the field.

Glaucoma has common prevalence among middle aged and the elderly. In Singapore, glaucoma affects over 50,000 people, or 3% of the population aged 50 and over. To determine long-term treatments for patients, regular monitoring of patients' eye pressure is necessary. However, current gold standard Goldmann Applanation Tonometry (also known as GAT) remains a clinical practice. GAT is expensive and requires specialised equipment. There may also be pain and discomfort from anaesthesia and corneal contact when performing GAT. Frequent hospital visits disrupt patients' daily routine too.

While there are handheld devices in the market that seek to provide a less complex and less expensive alternative to GAT equipment, handheld tonometer devices do require direct physical corneal contact and/or specialist's use.

Embodiments of the present invention seek to address at least one of the above problems.

### SUMMARY

In accordance with a first aspect of the present invention, there is provided a method of measuring intraocular pressure (IOP) of the eye comprising the steps of:
touching the eyelid with a pressure sensor array;
obtaining a spatiotemporal representation of pressure sensor stimulation of the pressure sensor array while touching the eyelid with the pressure sensor array; and
applying a machine learning model to classify the spatiotemporal representation into an IOP value.

In accordance with a second aspect of the present invention, there is provided a system for measuring intraocular pressure (IOP) of the eye comprising:
a pressure sensor array configured to the touch the eyelid; and
a processing module for obtaining a spatiotemporal representation of pressure sensor stimulation of the pressure sensor array while touching the eyelid with the pressure sensor array and for applying a machine learning model to classify the spatiotemporal representation into an IOP value.

### BRIEF DESCRIPTION OF THE DRAWINGS

Embodiments of the invention will be better understood and readily apparent to one of ordinary skill in the art from the following written description, by way of example only, and in conjunction with the drawings, in which:

FIG. 1A shows a design image illustrating a sensor device according to an example embodiment.

FIG. 1B shows a design image illustrating a sensor device according to an example embodiment.

FIG. 2 shows a schematic drawing illustrating operation of a sensor device according to an example embodiment in a test set-up.

FIG. 3B shows an average confusion matrix achieved with a sensor device according to an example embodiment, using Random Forest [1].

FIG. 3B shows an average confusion matrix achieved with a sensor device according to an example embodiment, using eXtreme Gradient Boosting [2].

FIG. 4 shows a representative spatiotemporal representation including pressure intensity information (color/shade coded) of the pressure sensor stimulation of a sensor device according to an example embodiment.

FIG. 5A is a schematic, cross-sectional drawing illustrating a fabricated a sensor array for a sensor device and method according to an example embodiment.

FIG. 5B is a schematic, plan view drawing illustrating the bottom electrode of a sensor array according to an example embodiment.

FIG. 6 shows a flowchart illustrating a method of measuring intraocular pressure (IOP) of the eye, according to an example embodiment.

FIG. 7 shows a schematic drawing illustrating a system for measuring intraocular pressure (IOP) of the eye, according to an example embodiment.

FIGS. 8A shows a schematic, perspective view drawing illustrating a sensor device and method according to an example embodiment.

FIGS. 8B shows a schematic, plan view drawing illustrating of the sensor device and method of FIG. 7A.

### DETAILED DESCRIPTION

Embodiments of the present invention provide a device that is applied on the eyelid, ideally non-invasive and free from direct contact with the cornea for intraocular pressure (IOP) sensing. Example embodiments are also applicable for patients with cornea irregularities. A machine learning algorithm according to an example embodiment promises an easy, fast, and accurate capture of eye pressure. Computed by the pre-trained AI model, embodiments of the present invention can preferably be independent of pressure applied and effect of eye variables.

In one embodiment, the present invention adopts a lightweight, wearable single-finger glove design with incorporated electronics into a smart watch display. A sensor array at the fingertip is connected to the smart watch display at the wrist through embedded flexible conductors in one embodiment, noting that wireless connection and/or cloud processing can be used in different example embodiments.

In another embodiment, the design could be in the form of a standalone handheld device with a pressure sensor array designed to actuate onto the eyelid for determination of the IOP. The device could control actuation of the pressure sensor array onto the eyelids with a maximum pressure limit to avoid overly high pressures onto the eyelids.

Embodiments of the present invention can allow users to test their IOP regularly and conveniently at home. In one example embodiment the user just needs to wear the glove with sensor placed at the fingertip. Specifically, after clicking the ‘start’ button on the smart watch the user then presses the fingertip upon the centre of the eyelid until hearing (or otherwise receiving) a ‘test complete’ notification. The sensor on the fingertip employs a sensor architecture that can capture dynamic pressure information of the user's eye with sub-millisecond precision. A pre-trained AI model processes the tactile pressure map into real-time eye pressure value(s) and the value(s) is presented to the users on smart watch. Data can also be transmitted via Bluetooth to paired devices or uploaded to cloud to be accessed remotely by clinicians.

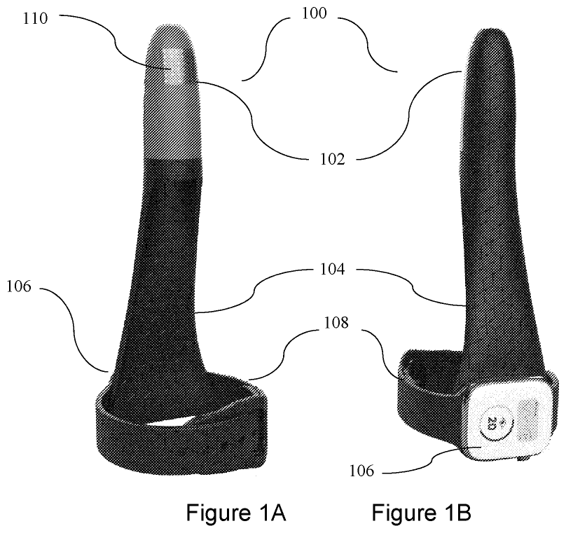

FIGS. 1A and B shows a design diagram of a sensor-based wearable device 100 for intraocular pressure (IOP) sensing according to an example embodiment. The device 100 includes a pressure sensor array 102 on a communication medium in the form of a single-finger glove 104 with embedded flexible conductors coupled to a receiver/processing unit in the form of a smart watch 106 integrated with the single-finger glove 104 and an adjustable wrist band 108. In a non-limiting example embodiment, the device 100 can be constructed as a sensor-based communication apparatus as described in US Patent Application Publication US 2020/0333881 A1, the contents of which are hereby incorporated by cross-reference.

Briefly, each pressure sensor e.g. 110 of the sensor array 102 is connected to a sensor node electrically attached to and embedded in the single-finger glove 104. The sensor nodes are associated with respective unique pulse signatures and are adapted to communicate with the respective pressure sensors e.g. 110. In this embodiment, each sensor node is integrally formed with the corresponding pressure sensor e.g. 110, although this may not be the case in other embodiments. Each pressure sensor e.g. 110 generates a sensory signal upon detecting a respective pressure stimulus, i.e. when the user touches the eyelid with the tip of the single-finger glove 104. In the present embodiment, each pressure sensor e.g. 110 is a tactile sensor responsive to a touch or pressure to generate the sensory signal. Each sensor node is triggered, upon receipt of the corresponding sensory signal from the respective pressure sensor e.g. 110, to transmit the associated unique pulse signature independently through the transmission medium in the form of the finger glove 104 with embedded flexible conductors shared by the sensor nodes. In other embodiments, the transmission medium can be any medium shared by the sensor nodes. For example, the transmission medium may be one capable of transmitting vibration/sound, optical, and/or magnetic field signals.

The unique pulse signatures are transmitted by the sensor nodes independently and asynchronously through the transmission medium in the form of the finger glove 104 are (or provide) a representation (e.g., a spatiotemporal representation) of a stimulus event associated with the stimuli detected by the corresponding pressure sensors e.g. 110. In this embodiment, the stimulus event is the tip of the single-finger glove 104, i.e. the sensor array 102, touching the (closed) eyelid. More particularly, the unique pulse signatures generated and transmitted by the respective sensor nodes collectively serve as a basis for acquisition of a spatiotemporal representation of the stimulus event associated with the pressure stimuli detected by the corresponding sensors e.g. 110. With knowledge of locations of the pressure sensors e.g. 110 and the respective times of triggering of the associated sensor nodes (i.e. of pressure detection by the sensors e.g. 110), a spatiotemporal representation of the pressure stimulus event can be accurately rendered. That is, the unique pulse signatures transmitted in association with a pressure stimulus event carry or preserve information temporally descriptive of detection of the respective pressure stimuli by the respective sensors e.g. 110. Combined with knowledge of locations (or relative locations) of the sensors e.g. 110, a spatiotemporal representation of pressure sensor stimulation can be rendered by the receiver/processing unit in the form of the smart watch 106. In an example embodiment, the intensity of the pressure stimulus for each individual sensor is also incorporated into the spatiotemporal representation of the pressure sensor stimulation, to create multidimensional sensor array data of the pressure sensor stimulation in the sensor array using the position, intensity, and temporal location of the stimulation.

It is noted that the present invention is not limited to the above described implementation for generating the pressure array data. Various other techniques may be used to generate, collect and process data from the sensor array to obtain the pressure array data of the pressure sensor stimulation in the sensor array representing the position and temporal location of the stimulation, and preferably including the intensity of the stimulation.

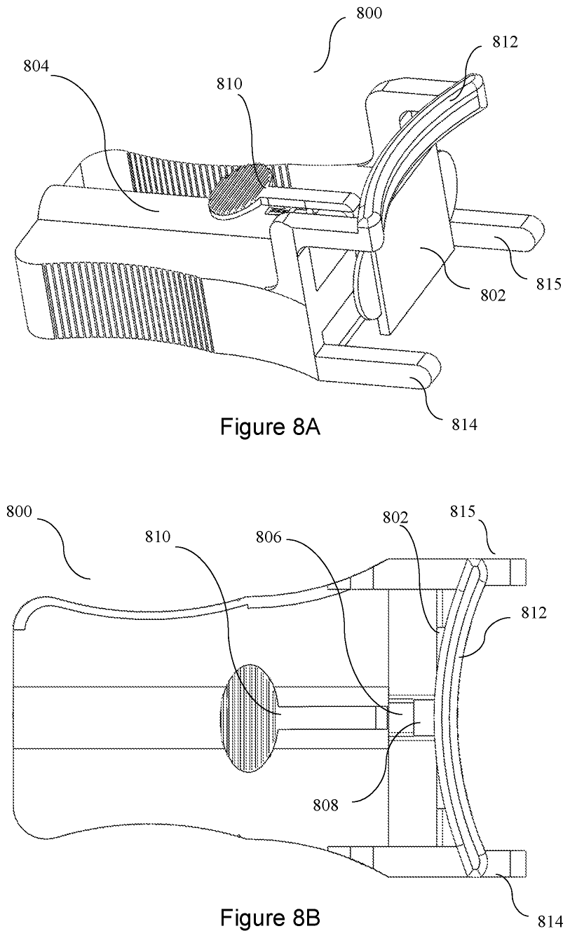

It is noted that the present invention is not limited to the implementation as a finger-tip sensor array carried on a glove or the like. Instead, various manual and/or automated actuators may be used in different embodiments for touching the eyelid with the sensor array. For example, the actuator may be implemented as a clinical desktop device for use with a chin/head rest for the patient. For example, FIGS. 8A and B show schematic drawings illustrating a sensor device 800 and method according to another non-limiting example embodiment. The sensor device 800 comprises a pressure sensor array pad 802 coupled to an actuator structure 804. In one non-limiting example implementation, the actuator structure 804 is automated using a motor (hidden inside the housing of the sensor device 800 FIGS. 8A and B) that drives a shaft 806 connected to a carrier 808 onto which the sensor array pad 802 is mounted. The motor is activated using the switch 810.

In operation, the sensor device 800, with the shaft 806/sensor array pad 802 in a retracted position, is placed in front of a person's eye, either by another person or by the person her-or himself. A forehead rest 812 and two cheek bone rests 814, 815 are provided to place the sensor device securely and at a desired distance from the person's eye. The forehead rest 812 and cheek bone rests 814, 815 are preferably adjustable to meet a person's individual requirements. When the sensor device 800 is securely placed in front of the eye, the actuator structure 804 is activated by pressing the switch 810. The motor is then controlled to move the shaft 806/sensor array pad 802 towards the eye with a programmed speed and displacement at a position where the sensor array pad 802 touches the eyelid. The displacement may be set relative to the position of the forehead rest 812 and/or cheek bone rests 814, 815, and/or one or more sensors may be incorporated in the actuator structure 804 for active feedback. The sensor array pad 802 is then held in place while touching the eyelid, and the measurements for obtaining the sensor array data of the pressure sensor stimulation in the sensor array pad 802 are performed. A processing unit (hidden insight the housing of the sensor device 800 in FIGS. 8A and B) coupled to sensor nodes (not shown) of the sensor array pad 802 perform the data processing, which may include the classification processing into the IOP value(s). Alternatively, the sensor array data may be transmitted to a remote processing unit, for example for the classification processing into the IOP value(s).

It has been found by the inventors that machine learning models can be applied to the rendered spatiotemporal representation of pressure sensor stimulation, optionally together with the intensity information of the pressure stimulus, for classification into an intraocular pressure (IOP) of the eye.

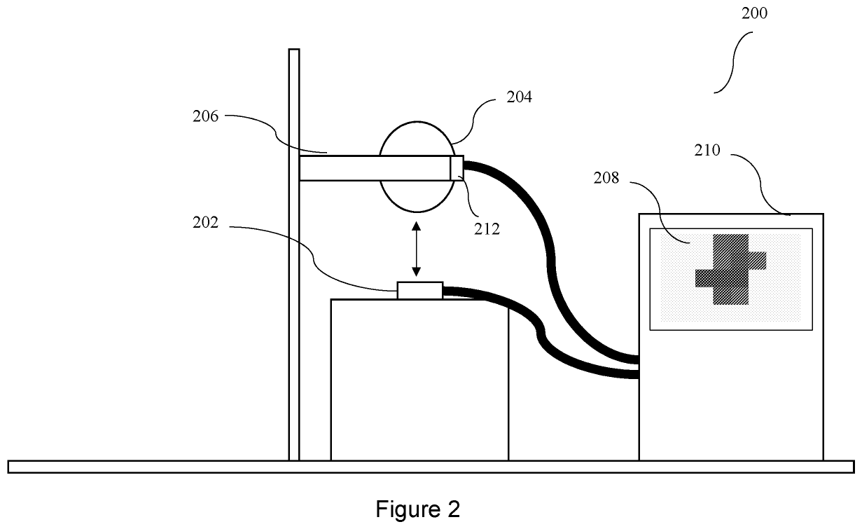

With reference to FIG. 2, a dataset was constructed using a proto-type sensor device 200 according to an example embodiment. An artificial eye model 204 was repeatedly pressed onto the sensor array 202 of the proto-type sensor device 200 according to an example embodiment using a z-axis stage 206, at a constant speed relative to the artificial eye model 204. The spatiotemporal representation 208 of the pressure sensor stimulation, including intensity information, and the IOP value that was set in the artificial eye model 204 was recorded for each iteration and machine learning was applied using a computer 210.

The duration of each contact was around 3 seconds. The artificial eye model 204 (controlled by the z-axis stage 206) was moved back to its original position after contact. The artificial eye model 206 is held in contact with the sensor array 202 by a target indentation depth controlled by the z-axis stage 206.

Different IOPs of the artificial eye model 204 were set by injecting different amount of water which is monitored by a water pressure sensor 212 connected to a computer 210 for measurement.

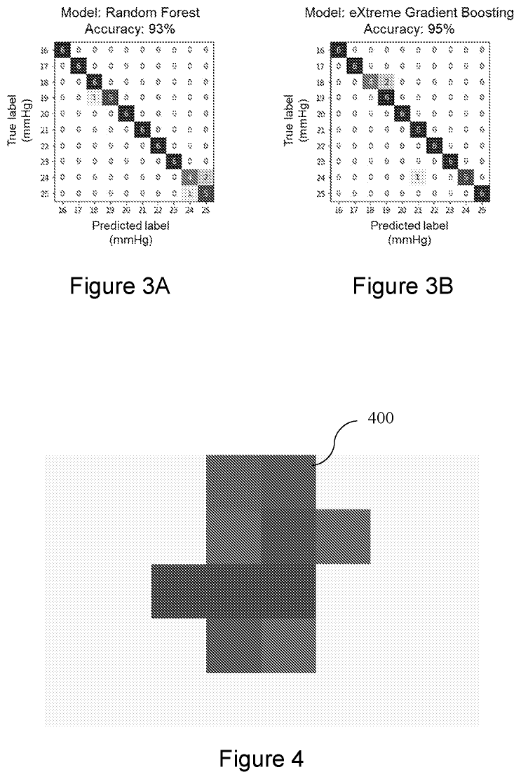

More specifically, the resultant output signals from the sensor nodes of the sensor array 202 and corresponding IOP values set in the artificial eye model 204 were recorded in the computer 208 and used for machine learning. The dataset was classified using two different models (Random Forest [1] and eXtreme Gradient Boosting [2]) to learn the unique feature of the pressure signals for IOP value classification. The models were trained repeatedly 10 times on random train-test (80%-20%) splits, and the average confusion matrix is shown in FIGS. 3A and 3B, respectively.

From the results shown in FIGS. 3A and 3B it can be seen that it is possible to classify IOP values from a spatial array of time-sequential pressure values with 93% and 95% accuracy, respectively, according to an example embodiment.

As described above, the pressure array data generated and transmitted by the respective sensor nodes of the proto-type sensor device according to an example embodiment collectively serve as a basis for acquisition of a spatiotemporal representation of the stimulus event associated with the pressure stimuli detected by the corresponding sensors when the sensor array is pressed onto the artificial eye model. With knowledge of locations of the individual pressure sensors relative to the surface of the artificial eye model and the respective stimulus event times of triggering of the associated sensor nodes, a spatiotemporal representation of the pressure stimulus event can thus be accurately rendered. That is, the unique pulse signatures transmitted in association with a pressure stimulus event carry or preserve information temporally descriptive of detection of the respective pressure stimuli by the respective sensors. A representative spatiotemporal representation 400 including pressure intensity information (color/shade coded) of the pressure sensor stimulation in the proto-type sensor device according to an example embodiment is shown in FIG. 4. It is noted that for the proto-type sensor device according to an example embodiment, only the sensors that are compressed will be activated and recorded as a pressure stimulus event e.g. 400.

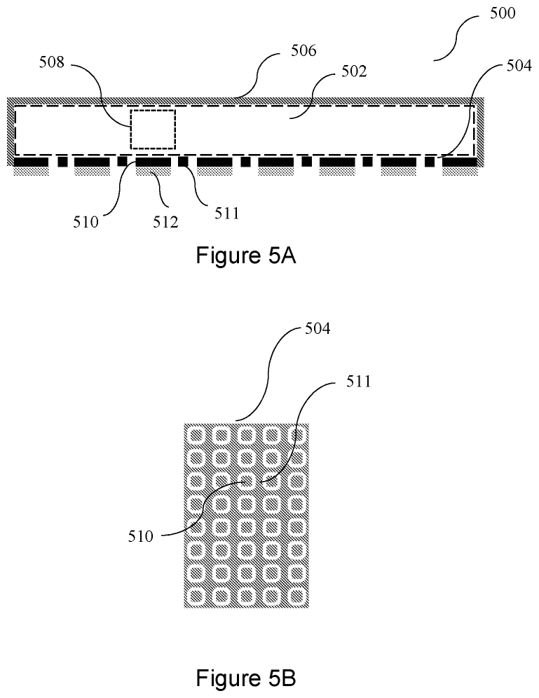

With reference to FIG. 5A, the sensor array 500 for a sensor device and method according to an example embodiment was fabricated by attaching a pressure-sensitive foil 502, e.g. made from a piezo-resistive material such as carbon impregnated carbon composite films or other films whereby the electrical properties change with applied pressure, but not limited thereto, to an arrayed bottom electrode 504, e.g. made from a metal such as a printed circuit board (PCB) with exposed immersion gold contact, but not limited thereto, followed by encapsulation with a thin polymeric sheet 506, e.g. made from polyethylene terephthalate (PET), but not limited thereto.

FIG. 5B shows the top view of the bottom electrode 504 in the example embodiment. There are no top electrodes in this example embodiment, but the present invention is not limited thereto. The pressure response is extracted using the bottom planar electrode 504. Specifically, respective isolated electrode elements e.g. 510 and the common metal plane 511 form an array of respective pairs of terminal metals.

With reference again to FIG. 5A, when the sensor array 500 is subjected to the pressure exerted by the interaction with the artificial eye model (compare e.g. FIG. 2), effected region(s) e.g. 508 of the pressure-sensitive foil 502 form a conductive path between electrode element(s) e.g. 510 at the location of the region(s) e.g. 508 and the common metal plane 511 as a result of the pressure sensitive response. A pressure stimulus event can thus be recorded via the current/charge response extracted by the electrode element(s) e.g. 510 and the common metal plane 511 at the location of the region(s) e.g. 508. In this example, the electrode elements e.g. 510 are formed integrally with circuit elements e.g. 512, together functioning as respective sensor nodes for the generation of the unique pulse signatures for transmission to the processing module (not shown).

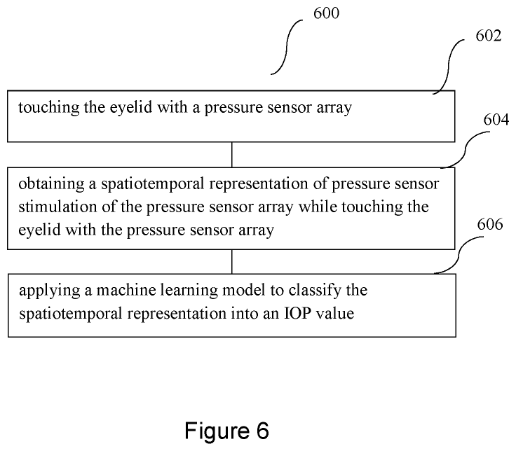

FIG. 6 shows a flowchart 600 illustrating a method of measuring intraocular pressure (IOP) of the eye, according to an example embodiment. At step 602, the eyelid is touched with a pressure sensor array. At step 604, a spatiotemporal representation of pressure sensor stimulation of the pressure sensor array while touching the eyelid with the pressure sensor array is obtained. At step 606, a machine learning model is applied to classify the spatiotemporal representation into an IOP value.

The method may comprise obtaining stimulation intensities measured by respective sensors of the sensor array. The machine learning model may be applied to classify the spatiotemporal representation including the stimulation intensities into the IOP value.

Touching the eyelid with the pressure sensor array may comprise carrying the pressure sensor array on a fingertip and touching the eyelid.

Touching the eyelid with the pressure sensor array may comprise using an actuator onto which the pressure sensor array is mounted.

Obtaining the spatiotemporal representation may comprise independently and asynchronously generating unique pulse signatures triggered by pressure stimuli events detected by the respective sensors of the pressure sensor array. The unique pulse signatures may be transmitted using wired or wireless communication for obtaining the spatiotemporal representation.

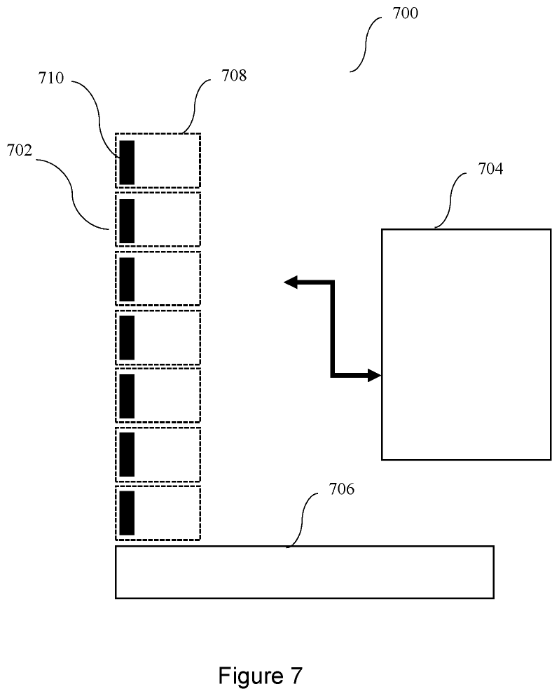

FIG. 7 shows a schematic drawing illustrating a system 700 for measuring intraocular pressure (IOP) of the eye, according to an example embodiment. The system 700 comprises a pressure sensor array 702 configured to the touch the eyelid; and a processing module 704 for obtaining a spatiotemporal representation of pressure sensor stimulation of the pressure sensor array 702 while touching the eyelid with the pressure sensor array 702 and for applying a machine learning model to classify the spatiotemporal representation into an IOP value.

The processing module 704 may be configured for obtaining stimulation intensities measured by respective sensors of the sensor array. The processing module 704 may be configured for applying the machine learning model to classify the spatiotemporal representation including the stimulation intensities into the IOP value.

The pressure sensor array 702 may be configured to be carried on a fingertip for touching the eyelid with the sensor array.

The system 700 may comprise an actuator 706 onto which the pressure sensor array 702 is mounted and configured for touching the eyelid with the sensor array 702.

The system 700 may comprise sensor nodes e.g. 708 for independently and asynchronously generating unique pulse signatures triggered by pressure stimuli events detected by the respective sensors e.g. 710 of the pressure sensor array 702 for obtaining the spatiotemporal representation. The sensor nodes e.g. 708 may be formed integrally with the respective sensors e.g. 710 or separately. The unique pulse signatures may be transmitted using wired or wireless communication between the sensor nodes e.g. 708 and the processing module 704.

The processing module 704 may be disposed locally relative to the sensor array 702 or remotely.

Aspects of the systems and methods described herein may be implemented as functionality programmed into any of a variety of circuitry, including programmable logic devices (PLDs), such as field programmable gate arrays (FPGAs), programmable array logic (PAL) devices, electrically programmable logic and memory devices and standard cell-based devices, as well as application specific integrated circuits (ASICs). Some other possibilities for implementing aspects of the system include: microcontrollers with memory (such as electronically erasable programmable read only memory (EEPROM)), embedded microprocessors, firmware, software, etc. Furthermore, aspects of the system may be embodied in microprocessors having software-based circuit emulation, discrete logic (sequential and combinatorial), custom devices, fuzzy (neural) logic, quantum devices, and hybrids of any of the above device types. Of course the underlying device technologies may be provided in a variety of component types, e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, etc.

The various functions or processes disclosed herein may be described as data and/or instructions embodied in various computer-readable media, in terms of their behavioral, register transfer, logic component, transistor, layout geometries, and/or other characteristics. Computer-readable media in which such formatted data and/or instructions may be embodied include, but are not limited to, non-volatile storage media in various forms (e.g., optical, magnetic or semiconductor storage media) and carrier waves that may be used to transfer such formatted data and/or instructions through wireless, optical, or wired signaling media or any combination thereof. When received into any of a variety of circuitry (e.g. a computer), such data and/or instruction may be processed by a processing entity (e.g., one or more processors).

The above description of illustrated embodiments of the systems and methods is not intended to be exhaustive or to limit the systems and methods to the precise forms disclosed. While specific embodiments of, and examples for, the systems components and methods are described herein for illustrative purposes, various equivalent modifications are possible within the scope of the systems, components and methods, as those skilled in the relevant art will recognize. The teachings of the systems and methods provided herein can be applied to other processing systems and methods, not only for the systems and methods described above.

It will be appreciated by a person skilled in the art that numerous variations and/or modifications may be made to the present invention as shown in the specific embodiments without departing from the spirit or scope of the invention as broadly described. The present embodiments are, therefore, to be considered in all respects to be illustrative and not restrictive. Also, the invention includes any combination of features described for different embodiments, including in the summary section, even if the feature or combination of features is not explicitly specified in the claims or the detailed description of the present embodiments.

In general, in the following claims, the terms used should not be construed to limit the systems and methods to the specific embodiments disclosed in the specification and the claims, but should be construed to include all processing systems that operate under the claims. Accordingly, the systems and methods are not limited by the disclosure, but instead the scope of the systems and methods is to be determined entirely by the claims.

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list and any combination of the items in the list.

### References

[1] Breiman, L. Random Forests. Machine Learning 45, 5-32 (2001). https://doi.org/10.1023/A: 1010933404324
[2] Chen Tianqi and Guestrin Carlos. 2016. XGBoost: A scalable tree boosting system. In Proceedings of the 22nd ACM SIGKDD International Conference on Knowledge Discovery and Data Mining. 785-794.

1. A method of measuring intraocular pressure (IOP) of the eye comprising the steps of:
touching the eyelid with a pressure sensor array;
obtaining a spatiotemporal representation of pressure sensor stimulation of the pressure sensor array while touching the eyelid with the pressure sensor array; and
applying a machine learning model to classify the spatiotemporal representation into an IOP value.

2. The method of claim 1, further comprising obtaining stimulation intensities measured by respective sensors of the sensor array.

3. The method of claim 2, wherein the machine learning model is applied to classify the spatiotemporal representation including the stimulation intensities into the IOP value.

4. The method of claim 1, wherein touching the eyelid with the pressure sensor array comprises carrying the pressure sensor array on a fingertip and touching the eyelid.

5. The method of claim 1, wherein touching the eyelid with the pressure sensor array comprises using an actuator onto which the pressure sensor array is mounted.

6. The method of claim 1, wherein obtaining the spatiotemporal representation comprises independently and asynchronously generating unique pulse signatures triggered by pressure stimuli events detected by the respective sensors of the pressure sensor array.

7. The method of claim 6, wherein the unique pulse signatures are transmitted using wired or wireless communication for obtaining the spatiotemporal representation.

8. A system for measuring intraocular pressure (IOP) of the eye comprising:
a pressure sensor array configured to the touch the eyelid; and
a processing module for obtaining a spatiotemporal representation of pressure sensor stimulation of the pressure sensor array while touching the eyelid with the pressure sensor array and for applying a machine learning model to classify the spatiotemporal representation into an IOP value.

9. The system of claim 8, wherein the processing module is configured for obtaining stimulation intensities measured by respective sensors of the sensor array.

10. The system of claim 9, wherein the processing module is configured for applying the machine learning model to classify the spatiotemporal representation including the stimulation intensities into the IOP value.

11. The system of claim 8, wherein the pressure sensor array is configured to be carried on a fingertip for touching the eyelid with the sensor array.

12. The system of claim 8, comprising an actuator onto which the pressure sensor array is mounted and configured for touching the eyelid with the sensor array.

13. The system of claim 8, comprising sensor nodes for independently and asynchronously generating unique pulse signatures triggered by pressure stimuli events detected by the respective sensors of the pressure sensor array for obtaining the spatiotemporal representation.

14. The system of claim 13, wherein the sensor nodes are formed integrally with the respective sensors or separately.

15. The system of claim 13, wherein the unique pulse signatures are transmitted using wired or wireless communication between the sensor nodes and the processing module.

16. The system of claim 8, wherein the processing module is disposed locally relative to the sensor array or remotely.
